(19) United States
(12) Patent Application Publication
(10) Pub. No.: US 2009/0176860 A1
(43) Pub. Date: Jul. 9, 2009

(54) ZOFENOPRIL CALCIUM

(76) Inventors: Vinayak G. Gore, Panvel (IN); Ashok D. Pehere, Panvel (IN); Avinash C. Gaikwad, Panvel (IN); Priyesh S. Vijaykar, Panvel (IN)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/277,841

(22) Filed: Nov. 25, 2008

Related U.S. Application Data
(63) Continuation of application No. PCT/GB2007/050296, filed on May 25, 2007.

(30) Foreign Application Priority Data
May 26, 2006 (IN) 815/MUM/2006

(51) Int. Cl. A61K 31/40; C07D 207/16; A61P 9/10

(52) U.S. Cl. 514/423; 548/533

(57) ABSTRACT

The present invention relates to zofenopril calcium form A substantially free of other forms of zofenopril calcium and to zofenopril calcium having a chemical purity of more than 98.5%. The present invention also relates to methods of preparing such zofenopril calcium. It further relates to compositions comprising such zofenopril calcium. Zofenopril calcium, (4S)-1-[(2S)-3-benzoylthio-2-methylpropionyl]-4-(phenylthio)-L-proline calcium salt, has the following structure:

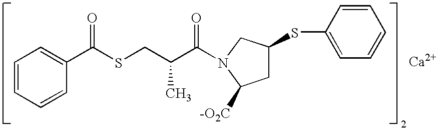

### CROSS-REFERENCE TO RELATED APPLICATIONS

This application is a continuation of International Patent Application No. PCT/GB2007/050296, filed on May 25, 2007, which claims priority to Indian Patent Application No. 815/mum/2006, filed on May 26, 2006, the entire contents of both of which are incorporated herein by reference.

### FIELD OF THE INVENTION

The present invention relates to zofenopril calcium form A substantially free of other forms of zofenopril calcium and to zofenopril calcium having a chemical purity of more than 98.5%. The present invention also relates to methods of preparing such zofenopril calcium. It further relates to compositions comprising such zofenopril calcium. Zofenopril calcium, (4S)-1-[(2S)-3-benzoylthio-2-methylpropionyl]-4-(phenylthio)-L-proline calcium salt, has the following structure:

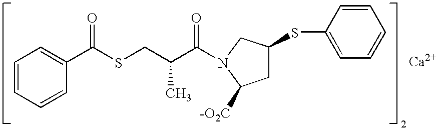

### BACKGROUND OF THE INVENTION

The manufacturing process for many pharmaceuticals is hindered by the fact that the organic compound which is the active drug substance is difficult to handle during the manufacturing process. These difficulties can lead to undesirable properties being imparted to the final drug or dosage form. In addition, it can be difficult to control the polymorphic form of the active drug substance throughout the manufacturing process.

For pharmaceuticals in which the active ingredient can exist in more than one polymorphic form, it is particularly important to ensure that the manufacturing process for the active ingredient affords a single polymorph with a consistent level of polymorphic purity. If the process leads to a polymorph with varying degrees of polymorphic purity and/or where the process does not control polymorphic interconversion, serious problems with dissolution and/or bioavailability can arise in the finished pharmaceutical formulation comprising the active ingredient. Zofenopril calcium, (4S)-1-[(2S)-3-benzoylthio-2-methylpropionyl]-4-(phenylthio)-L-proline calcium salt, is a non-peptidic, orally active, long acting ACE inhibitor. It is marketed for the treatment of hypertension under the trade name Zoprace®.

Zofenopril and methods for its preparation were first described in U.S. Pat. No. 4,316,906. Two polymorphs of zofenopril calcium, forms A and B, were disclosed in U.S. Pat. No. 6,515,012 and international patent application WO 00/07984. It was also disclosed in U.S. Pat. No. 6,515,012 and WO 00/07984 that polymorph A is much more industrially suitable than form B. Polymorph forms A and B of zofenopril calcium disclosed in U.S. Pat. No. 6,515,012 and WO 00/07984 show the following XRD patterns:

TABLE 1
XRD Comparison Position [2θ values]

| Polymorph A | Polymorph B |
|---|---|
| 4.3 | |
| | 4.8 |
| 7.4 | |
| 8.7 | |
| 10.1 | |
| 10.8 | |
| 11.7 | |
| 13.0 | |
| 14.8 | |
| | 15.6 |
| 16.0 | |
| 17.2 | |
| | 17.5 |
| 18.2 | |
| | 18.5 |
| 19.0 | |
| | 19.4 |
| 20.0 | |
| | 20.5 |
| | 21.4 |
| 21.7 | |
| | 21.8 |
| | 23.1 |
| 23.5 | |
| 24.6 | 24.6 |

One method for the preparation of zofenopril calcium as disclosed in U.S. Pat. No. 4,316,906 mainly yields polymorph A, but there is always contamination with polymorph B to varying degrees (never below 20%). An alternative synthesis described in U.S. Pat. No. 4,316,906 affords a partially amorphous product, with very variable characteristics, in which polymorph A, when present, is in concentrations much lower than those obtained in the preceding process.

A process for the preparation of supposedly pure form A is disclosed in U.S. Pat. No. 6,515,012 and WO 00/07984, and is summarised below:
(a) reaction of S(−)-3-benzoylthio-2-methyl-propanoic acid chloride and cis-4-phenylthio-L-proline in water at pH ranging from 9.0 to 9.5 and recovery of zofenopril in the acidic form,
(b) salification of acid zofenopril with a potassium salt in alcoholic solution and recovery of the resulting potassium salt,
(c) conversion of the potassium salt to calcium salt by addition of an aqueous solution of zofenopril potassium salt to a calcium chloride aqueous solution at up to 90° C. with simultaneous seeding to promote the precipitation of polymorph A.

However, the above method for the preparation of polymorph A has the following drawbacks:
The reaction is carried out at a relatively high temperature (up to 90° C.) at which interconversion of the polymorphs is possible.
Polymorph A can be obtained from the above process, but the product is always contaminated with polymorph B.
The chemical HPLC purity of the zofenopril obtained is only between 97-98%.

Therefore there remains a need for an improved process for the preparation of zofenopril calcium form A with high polymorphic purity. There also remains a need for an improved process for the preparation of zofenopril calcium with high chemical purity.

### OBJECT OF THE INVENTION

It is an object of the present invention to provide a process for the preparation of zofenopril calcium polymorph A of high optical purity, in particular zofenopril calcium form A which is substantially free of form B or other polymorphs. It is also an object of the present invention to provide a process for the preparation of zofenopril calcium of high chemical purity.

It is a further object of the present invention to provide zofenopril calcium polymorph A of high optical purity. It is also an object of the present invention to provide zofenopril calcium of high chemical purity.

It is a further object of the present invention to provide compositions comprising zofenopril calcium polymorph A of high optical purity. It is also an object of the present invention to provide compositions comprising zofenopril calcium of high chemical purity.

It is also an object of the present invention to provide zofenopril calcium in a solid crystalline form that affords the compound improved handling properties (such as increased resistance to compression and/or micronisation) and/or improved properties as a pharmaceutical agent, and enables strict control of the purity and/or polymorphic form during manufacturing.

### SUMMARY OF THE INVENTION

A first aspect of the present invention provides zofenopril calcium form A, substantially free of other forms of zofenopril calcium. Zofenopril calcium form A shows XRD peaks at substantially the following scattering angles 2θ: 4.3, 7.4, 8.7, 10.1, 10.8, 11.7, 13.0, 14.8, 16.0, 17.2, 18.2, 19.0, 20.0, 21.7, 23.5, and 24.6.

In the context of the present application, the term ‘substantially free’ of other forms of zofenopril calcium means that the zofenopril calcium form A comprises less than 6% of other crystalline or amorphous forms of zofenopril calcium, preferably less than 5%, preferably less than 4%, preferably less than 3%, preferably less than 2%, preferably less than 1%.

The first aspect of the present invention also provides zofenopril calcium having a chemical purity of more than 98.5%, preferably more than 99%, preferably more than 99.3%, as measured by HPLC.

A second aspect of the present invention provides a pharmaceutical composition comprising zofenopril calcium according to the first aspect of the present invention.

A third aspect of the present invention provides use of zofenopril calcium according to the first aspect of the present invention, for the manufacture of a medicament for the treatment of hypertension or another angiotensin-mediated condition.

A fourth aspect of the present invention provides a method of treating hypertension or another angiotensin-mediated condition, comprising administering a therapeutically effective amount of zofenopril calcium according to the first aspect of the present invention to a patient in need thereof.

A fifth aspect of the present invention provides a method of preparing zofenopril calcium according to the first aspect of the present invention. Preferably the method comprises the use of zofenopril potassium or zofenopril dicyclohexylamine. Preferably the method comprises the use of calcium chloride. Preferably the method comprises the use of an organic solvent/water system.

The fifth aspect of the present invention also provides a method of preparing zofenopril calcium, comprising the step of reacting a zofenopril salt other than zofenopril calcium with an aqueous solution of a calcium salt in an organic solvent/water system.

The fifth aspect of the present invention also provides a method of preparing zofenopril calcium, comprising the steps of:
a) combining an aqueous solution of a calcium salt and a solution of a zofenopril salt other than zofenopril calcium in an organic, water miscible solvent,
b) maintaining the resulting mixture until zofenopril calcium precipitates, and
c) separating the zofenopril calcium from the suspension.

Preferably the calcium salt used in step (a) is soluble in water, preferably readily soluble in water. Calcium salts which are soluble in water include, but are not limited to, calcium chloride, calcium bromide, calcium iodide, calcium formate, calcium acetate, calcium propionate, calcium butyrate, calcium isobutyrate, calcium α-methyl butyrate, and calcium oxide. A preferred calcium salt used in step (a) is calcium chloride.

Preferably the zofenopril salt used in step (a) is a lithium, sodium, potassium, magnesium, amine or amino acid salt of zofenopril. Amine salts of zofenopril include, but are not limited to, the dibenzylamine, N,N-dibenzylethylenediamine, methylamine, dimethylamine, trimethylamine, ethylamine, diethylamine, triethylamine, N-ethylmethylamine, t-butylamine, N,N-diisopropylethylamine, N,N-diisopropylmethylamine, procaine, N-ethylpiperidine, cyclohexylamine, dicyclohexylamine, and 1-adamantanamine salt. Amino acid salts of zofenopril include, but are not limited to, the arginine and lysine salt. Preferred zofenopril salts used in step (a) are zofenopril potassium and zofenopril dicyclohexylamine.

Organic, water miscible solvents include, but are not limited to, alcohols, aliphatic ketones (such as acetone or ethyl methyl ketone), acetonitrile, formamide, N,N-dimethylformamide, dimethylsulfoxide, sulfolane, diglyme, dioxane, tetrahydrofuran, and N-methylpyrrolidine. Preferably the organic, water miscible solvent is a lower alkyl alcohol. Lower alkyl alcohols include, but are not limited to, methanol, ethanol, n-propanol, isopropanol and n-butanol. Preferably the organic, water miscible solvent is methanol.

In step (a), the aqueous solution of a calcium salt and the solution of a zofenopril salt in an organic, water miscible solvent are preferably combined in a water organic solvent ratio of 1:1 to 1:10, preferably 1:1 to 1:5, more preferably 1:1 to 1:3.

Preferably the combining of step (a) is carried out at a temperature of up to 85° C., preferably at a temperature of 55-85° C., preferably 55-70° C., preferably 55-60° C.

Preferably the maintaining of step (b) is carried out with stirring and/or for at least one hour. Preferably the maintaining of step (b) is carried out at a temperature of up to 85° C., preferably at a temperature of 55-85° C., preferably 55-70° C., preferably 55-60° C.

Preferably the method is carried out without seeding zofenopril calcium form A.

The separating of step (c) may be achieved by filtering the suspension. Preferably the separating of step (c) is carried out at a temperature of up to 85° C., preferably up to 70° C., preferably up to 60° C., preferably up to 55° C.

The separated zofenopril calcium may be purified by washing with water, preferably until the zofenopril calcium is substantially free from chloride ions.

Alternatively or additionally, the separated zofenopril calcium may be purified by washing with methanol. The washing with methanol may be achieved by stirring the zofenopril calcium in methanol and filtering.

The separated zofenopril calcium may be dried under reduced pressure at a temperature of up to 85° C., preferably up to 70° C., preferably up to 60° C., preferably up to 50° C.

### DETAILED DESCRIPTION OF THE PRESENT INVENTION

Zofenopril calcium polymorph A obtained by the prior art processes is always contaminated with polymorph B.

The present invention provides zofenopril calcium form A substantially free of other polymorphs.

The present invention provides polymorph form A that has a level of polymorph form B or other polymorphs at a level of less than 10%, preferably less than 8%, more preferably less than 5%, even more preferably less than 3%, and most preferably less than 1%.

The present invention comprises the use of the dicyclohexylamine (DCHA) salt of the zofenopril acid.

The present invention comprises the use of the potassium salt of the zofenopril acid in an alcohol/water solvent system at relatively mild temperature conditions, which exercise strict control on polymorph composition thereby preventing the interconversion of polymorphs. The alcohol used can be any lower alkyl alcohol, but is preferably methanol.

The present invention yields zofenopril calcium form A of very good HPLC (chemical) purity (more than 98.5%, preferably more than 99%, preferably more than 99.3%).

The present invention provides pharmaceutical compositions comprising pure polymorph A of zofenopril calcium.

The pharmaceutical composition of the present invention is preferably solid and can comprise a compound in accordance with the invention in addition to one or more conventional pharmaceutically acceptable excipient(s). Preferred dosage forms in accordance with the invention include tablets, capsules and the like.

The compounds of the present invention may also be useful as precursors to other novel or known polymorphic forms of zofenopril that may be useful in the preparation of pharmaceutical products. Alternatively, the compounds in accordance with the present invention may be used to prepare other desired polymorphic forms of zofenopril in a more controllable manner.

The present invention is illustrated, but in no way limited, by the following examples.

### Example 1

An aqueous solution of calcium chloride was added to a methanolic solution (methanol: water=1:1) of DCHA salt of zofenopril acid at 80-85° C. and the resulting suspension was stirred for 3 hours. The resulting suspension was filtered off at 55° C. and the product was washed with water until free from chloride ions. The wet cake was stirred in methanol and filtered and the wet solids were dried under reduced pressure at 50° C. until a constant weight was obtained.

Purity=99.4% (HPLC).

Melting point=235-246° C.; DSC endotherm at 257° C.

The XRPD pattern of the product thus obtained matched with the reported polymorph A (WO 00/07984).

The product obtained had only traces of form B detectable by XRPD. The detection limit of the XRPD instrument used was 3%.

### Example 2

An aqueous solution of calcium chloride was added to a methanolic solution (methanol: water=3:1) of DCHA salt of zofenopril acid at 80-85° C. and the resulting suspension was stirred for 3 hours. The resulting suspension was filtered off at 55° C. and the product was washed with water until free from chloride ions. The wet cake was stirred in methanol and filtered and the wet solids were dried under reduced pressure at 50° C. until a constant weight was obtained.

Purity=99.39% (HPLC).

Melting point=235-246° C.; DSC endotherm at 254° C.

The XRPD pattern of the product thus obtained matched with the reported polymorph A (WO 00/07984).

The product obtained had only traces of form B detectable by XRPD. The detection limit of the XRPD instrument used was 3%.

### Example 3

An aqueous solution of calcium chloride was added to a methanolic solution (methanol: water=1:1) of DCHA salt of zofenopril acid at 55-60° C. and the resulting suspension was stirred for 3 hours. The resulting suspension was filtered off at 55° C. and the product was washed with water until free from chloride ions. The wet cake was stirred in methanol and filtered and the wet solids were dried under reduced pressure at 50° C. until a constant weight was obtained.

Purity=99.24% (HPLC).

Melting point=235-246° C.; DSC endotherm at 255.7° C.

The XRPD pattern of the product thus obtained matched with the reported polymorph A (WO 00/07984).

The product obtained had only traces of form B detectable by XRPD. The detection limit of the XRPD instrument used was 3%.

### Example 4

An aqueous solution of calcium chloride was added to a methanolic solution (methanol: water=1:1) of the potassium salt of zofenopril acid at 55-60° C. and the resulting suspension was stirred for 3 hours. The resulting suspension was filtered off at 55° C. and the product was washed with water until free from chloride ions. The wet cake was stirred in methanol and filtered and the wet solids were dried under reduced pressure at 50° C. until a constant weight was obtained.

Purity=99.43% (HPLC).

Melting point=235-246° C.; DSC endotherm at 258° C.

The XRPD pattern of the product thus obtained matched with the reported polymorph A (WO 00/07984)

The product obtained had no trace of form B detectable by XRPD. The detection limit of the XRPD instrument used was 3%.

1. A zofenopril calcium, comprising zofenopril calcium form A that is substantially free of other forms of zofenopril calcium.

2. The zofenopril calcium form A as claimed in claim 1, wherein the zofenopril calcium form A shows XRD peaks at substantially the following scattering angles 2θ: 4.3, 7.4, 8.7, 10.1, 10.8, 11.7, 13.0, 14.8, 16.0, 17.2, 18.2, 19.0, 20.0, 21.7, 23.5, and 24.6.

3. The zofenopril calcium form A as claimed in claim 1, wherein the zofenopril calcium form A comprises less than 6% of other forms of zofenopril calcium.

4. A zofenopril calcium, wherein the zofenopril calcium has a chemical purity of more than 98.5%.

5. A pharmaceutical composition comprising zofenopril calcium as claimed in claim 1 and a pharmaceutically acceptable excipient.

6. A pharmaceutical composition comprising zofenopril calcium as claimed in claim 4 and a pharmaceutically acceptable excipient.

7. A method of treating hypertension or an angiotensin-mediated condition, comprising administering a therapeutically effective amount of zofenopril calcium as claimed in claim 1 to a patient in need thereof.

8. A method of treating hypertension or an angiotensin-mediated condition, comprising administering a therapeutically effective amount of zofenopril calcium as claimed in claim 4 to a patient in need thereof.

9. A method of preparing zofenopril calcium, comprising the steps of:
(a) combining an aqueous solution of a calcium salt and a solution of a zofenopril salt other than zofenopril calcium in an organic, water miscible solvent,
(b) maintaining the resulting mixture until zofenopril calcium precipitates, and
(c) separating the zofenopril calcium from the suspension.

10. The method as claimed in claim 9, wherein the zofenopril calcium is of form
A and is substantially free of other forms of zofenopril calcium.

11. The method as claimed in claim 9, wherein the zofenopril calcium has a chemical purity of more than 98.5%.

12. The method as claimed in claim 9, wherein the calcium salt used in step (a) is:
(i) soluble in water; and/or
(ii) calcium chloride, calcium bromide, calcium iodide, calcium formate, calcium acetate, calcium propionate, calcium butyrate, calcium isobutyrate, calcium α-methyl butyrate, or calcium oxide; and/or
(iii) calcium chloride.

13. The method as claimed in claim 9, wherein the zofenopril salt used in step (a) is:
(i) a lithium, sodium, potassium, magnesium, amine or amino acid salt of zofenopril; and/or
(ii) zofenopril potassium or zofenopril dicyclohexylamine.

14. The method as claimed in claim 9, wherein the organic, water miscible solvent is:
(i) an alcohol, an aliphatic ketone, acetonitrile, formamide, N,N-dimethylformamide, dimethylsulfoxide, sulfolane, diglyme, dioxane, tetrahydrofuran, or N-methylpyrrolidine; and/or
(ii) a lower alkyl alcohol; and/or
(iii) methanol.

15. The method as claimed in claim 9, wherein:
(i) in step (a) the aqueous solution of a calcium salt and the solution of a zofenopril salt in an organic, water miscible solvent are combined in a water organic solvent ratio of 1:1 to 1:10; and/or
(ii) the combining of step (a) is carried out at a temperature of up to 85° C.;
and/or
(iii) the maintaining of step (b) is carried out with stirring; and/or
(iv) the maintaining of step (b) is carried out for at least one hour; and/or
(v) the maintaining of step (b) is carried out at a temperature of up to 85° C.;
and/or
(vi) the method is carried out without seeding zofenopril calcium form A; and/or
(vii) the separating of step (c) is achieved by filtering the suspension; and/or
(viii) the separating of step (c) is carried out at a temperature of up to 85° C.;
and/or
(ix) the separated zofenopril calcium is purified by washing with water or methanol; and/or
(x) The separated zofenopril calcium is purified by washing with methanol by stirring the zofenopril calcium in methanol and filtering; and/or
(xi) the separated zofenopril calcium is dried under reduced pressure at a temperature of up to 85° C.
